SEQUENCE LISTING (2 sequences)

SEQ ID NO: 1 - protein, 672 aa, Arabidopsis thaliana
  DOMAIN (268)..(346): RAD51-binding domain
MCGSVVSDSEREFEFHFWGFGHLGKEEERIMAGKRSSPFSSPLLRPPPEFLNVKEEGETETPCWRKEVDENLKRLQSLLFGADKFLEKSDFSSAQILGLRLLGFLDSRSVTDADRDFIGPIRREVASKIDLALEGLVSDSDRKAFELANTAPGAIFGSKGGFDVEKIKQSKYFGFHVSQSNGKGVKEMLIPKAPKSMMQAKLTSLYGNSIGKPDNQRKTSVNNQDRASDECVIVERSHGFGFGTKRPHAETSSLANDGEVKEDGAPNGFVSAKIKLEMDVRQKRGSTESPSSCLSPQSDKNALGRGYGSRSGGLRRGYRSNFVPPVKTNGNNVGNLTSRIGGKTDDALDDSTRTCLEMLCGPDGELPEKLRNLEPRLIEHVSNEIMDRDPNVRWDDIAGLEHAKKCVTEMVIWPLLRPDIFKGCRSPGKGLLLFGPPGTGKTMIGKAIAGEAKATFFYISASSLTSKWIGEGEKLVRALFGVASCRQPAVIFVDEIDSLLSQRKSDGEHESSRRLKTQFLIEMEGFDSGSEQILLIGATNRPQELDEAARRRLTKRLYIPLPSSEARAWIIQNLLKKDGLFTLSDDDMNIICNLTEGYSGSDMKNLVKDATMGPLREALKRGIDITNLTKDDMRLVTLQDFKDALQEVRPSVSQNELGIYENWNNQFGSLSL

SEQ ID NO: 2 - protein, 1390 aa, Arabidopsis thaliana
  DOMAIN (129)..(272): DEXDc helicase domain
MGSRVPIETIEEDGEFDWEAAVKEIDLACLKTTNASSSSSSHFTPLANPPITANLTKPPAKRQSTLDKFIGRTEHKPENHQVVSECGVNDNDNSPLVGIDPEAAKTWIYPGFVPLRDYQFAITKTALFSNTLVALPTGLGKTLIAAVVMYNYFRWFPQGKIVFAAPSRPLVMQQIEACHNIVGIPQEWTIDLTGQTCPSKRAFLWKSKRVFFVTPQVLEKDIQSGTCLTNYLVCLVIDEAHRALGNYSYCVVVRELMAVPIQLRILALTATPGSKTQAIQGIIDNLQISTLEYRNESDHDVCPYVHDRKLEVIEVPLGQDADDVSKRLFHVIRPYAVRLKNFGVNLNRDIQTLSPHEVLMARDKFRQAPLPGLPHVNHGDVESCFAALITLYHIRKLLSSHGIRPAYEMLEEKLKEGPFARLMSKNEDIRMTKLLMQQRLSHGAPSPKLSKMLEILVDHFKVKDPKTSRVIIFSNFRGSVRDIMNALSNIGDMVKATEFIGQSSGKTLKGQSQKIQQAVLEKFRAGGFNVIVATSIGEEGLDIMEVDLVICFDANVSPLRMIQRMGRTGRKNNGRPLLVLACEGSEKNSYMRKQASGRAIKKHMRNGGTNSFNFHPSPRMIPHVYKPEVQHVEFSIKQFVPRGKKLQEEYATETPAFQKKLTPAETHMLAKYYNNPDEEKLRVSLIAFPHFQTLPSKVHKVMHSRQTGMLIDAMQHLQEPTFSEQSKSFFTEFRAPLGEREELDTGLRVTNDPKDLHSVRDLEVNTSQRKAKQVESPTSTLETTEKDYEESSPTHRYLFSSECASVDTLGNVFVMPVPLLFFPNVLESDNTPLPKTEKQHSCRNTSHIDLVPVDTSEKHRQDNISCKLKERFSPDGASETLETHSLVKRNSTRVGEDDVANSVGEIVLSSDEDDCEGLELSPRLTNFIKSGIVPESPVYDQGEANREEDLEFPQLSSPMRFSNELAGESSFPERKVQHKCNDYNIVSTTTELRTPQKEVGLANGTECLAVSPIPEDWRTPLANLTNTNSSARKDWRVSSGEKLETLRQPRKLKRLRRLGDCSSAVKENYPGITEADHIRSRSRGKKHIRVKWKSAESWFTLSSVAFALVTKKKKMIMDDDVQVFIDEEAEVSSGAEMSADENEDVTGDSFEDSFIDDGTMPTANTQAESGKVDMMAVYRYIQPKISFFYCEVNELIKNHKVSFHRRSLLSQSPLPARFRDLAASSLSPYSAGPLTRINESRSDSDKSLSSLRTPKTTNSESNQDAMMIGNLSVVQISSDSRKRKFSLCNSANAPVINLESKFAAHAQATEKESHEGVRSNAGALEYNDDDDDAFFATLDFDAMEAQATLLLSKQRSEAKEKEDATVIPNPGMQRSDGMEKDAPSFDLGLW

(19) United States
(12) Patent Application Publication
(10) Pub. No.: US 2016/0369287 A1
(43) Pub. Date: Dec. 22, 2016

(54) INCREASED MEIOTIC RECOMBINATION IN PLANTS BY INHIBITION OF THE FIDG PROTEIN

(71) Applicant: INSTITUT NATIONAL DE LA RECHERCHE AGRONOMIQUE, Paris (FR)

(72) Inventors: Raphaël MERCIER, Fontenay-Le-Fleury (FR); Chloé GIRARD, Paris (FR); Wayne CRISMANI, Vanves (FR)

(21) Appl. No.: 14/901,954

(22) PCT Filed: Jun. 30, 2014
(86) PCT No.: PCT/IB2014/062724

§ 371 Date: Dec. 29, 2015

(30) Foreign Application Priority Data
Jul. 3, 2013 (FR) 1356522

(51) Int. Cl. C12N 15/82; C12N 9/14

(52) U.S. Cl. CPC C12N 15/8241; C12N 9/14; C12Y 306/01003; C12Y 306/04012; C12N 15/8218

(57) ABSTRACT

The invention relates to a method for increasing the frequency of meiotic recombination in plants, by inactivating the FIDG protein, in particular by mutagenesis or silencing of the FIDG gene encoding said protein. The present invention is useable in particular in the field of plant improvement and of genetic mapping.

The present invention relates to a method for increasing meiotic recombination in plants.

Meiotic recombination is an exchange of DNA between homologous chromosomes during meiosis; it occurs during the prophase of the first meiotic division. One of the products of recombination is crossing over, which leads to a reciprocal exchange of continuity between two homologous chromatids. This prophase (prophase I) comprises 5 successive stages: leptotene, zygotene, pachytene, diplotene and diakinesis. At the leptotene stage the chromosomes become individualized, each chromosome being made up of two sister chromatids resulting from the duplication which occurred before prophase. During the zygotene stage, the homologous chromosomes pair up, forming a structure known as “bivalent” which contains four chromatids, two maternal sister chromatids and two paternal sister chromatids, which are homologous to the maternal chromatids. At the pachytene stage, the chromosomes are completely paired, and recombination nodules form between the homologous chromatids which are tightly linked to one another by the synaptonemal complex (SC); at the diplotene stage, the SC gradually dissociates, and the homologous chromosomes begin to separate from one another, but remain attached at the chiasmas, which correspond to the sites of crossovers (COs). The chromosomes condense during diakinesis; the chiasmas remain until metaphase I, during which they maintain the pairing of the bivalents on either side of the equatorial plate.

Meiotic recombination is triggered by the formation of double-stranded breaks (DSBs) in one or other of the homologous chromatids, and result from the repairing of these breaks using, as template, a chromatid of the homologous chromosome.

The result of meiotic recombination is to cause a rearrangement of the alleles of paternal and maternal origin in the genome, thereby contributing to generating genetic diversity. It is therefore of particular interest in plant improvement programs (Wijnker & de Jong, Trends in Plant Science, 13, 640-646, 2008, Crismani et al., Journal of Experimental Botany, 64, 55-65, 2013). In particular, a control in recombination rate can make it possible to increase genetic mixing, and therefore the probabilities of obtaining new combinations of characteristics; it can also make it possible to facilitate the introgression of genes of interest, and also the genetic mapping and the positional cloning of genes of interest.

Various methods for controlling meiotic recombination have been proposed, based on the overexpression or the silencing of one or other of the very large number of genes identified as being involved or potentially involved in this mechanism. For example, PCT application WO/0208432 proposes the overexpression of the RAD51 protein, which is involved in homologous recombination, for stimulating meiotic recombination; application US 2004023388 proposes overexpressing an activator of meiotic recombination, chosen from: SPO11, MRE11, RAD50, XRS2NBS1, DMC1, RAD51, RPA, MSH4, MSHS, MLH1, RAD52, RAD54, TID1, RAD5S, RADS7, RADS9, a resolvase, a single-stranded DNA-binding protein, a protein involved in chromatin remodeling, or a protein of the synaptonemal complex, for increasing the frequency of recombination between homologous chromatids; PCT application WO 2004/016795 proposes increasing the recombination between homologous chromosomes expressing an SPO11 protein fused to a DNA-binding domain; PCT application WO 03/104451 proposes increasing the potential for recombination between homologous chromosomes by overexpression of a protein (MutS) involved in mismatch repair.

However, in most cases, the effect of these candidate genes on the formation of meiotic COs in planta has not been confirmed, and it is therefore necessary to identify other genes involved in this phenomenon.

One of the factors known to act on the meiotic recombination rate is the interference phenomenon: the formation of a CO at a site on the chromosome inhibits the formation of other COs close by. However, it has been shown that there are in fact 2 distinct pathways for meiotic CO formation (Hollingsworth & Brill, Genes Dev., 18,117-125, 2004; Berchowitz & Copenhaver, Current genomics, 11, 91-102, 2010). The first generates interfering COs known as type I COs (COI), and involves a set of genes collectively denoted as ZMM genes (ZIP1, ZIP2/SHOC1, ZIP3, HEI10, ZIP4, MER3, MSH4, MSH5, PTD) and also the MLH1 and MLH3 proteins; the second pathway generates non-interfering COs, known as type II COs (COII), and is dependent on the MUS81 gene.

The use of one and/or the other of these two pathways is variable from one organism to another. In higher plants, represented by the model plant Arabidopsis thaliana, the 2 pathways coexist, that of the type I COs being predominant; it has been observed that the inactivation of the AtMSH4, AtMSH5, AtMER3, SHOC1, PTD, AtHEI10 or AtZIP4 genes induces up to an 85% reduction in CO frequency (Higgins et al., 2004, mentioned above; Mercier et al., 2005, mentioned above; Chelysheva et al., PLoS Genet. 3, e83, 2007; Higgins et al., Plant J., 55, 28-39, 2008; Macaisne et al., Current Biology, 18, 1432-1437, 2008; Wijeratne et al., Molecular Biology of the cell, 17, 1331-43; Chelysheva et al., PLoS Genetics 8, e1002799, 2012). Some homologous chromosomes are as a result no longer paired in the form of bivalents, but appear in the form of univalents. This decrease in the number of bivalents is accompanied by a strong reduction in fertility.

Previously, the inventors identified a gene, known as FANCM (for “Fanconi Anemia Complementation Group M”), the inactivation of which compensated for the effects of that of AtMSH5, of SHOC1 or of AtZIP4, and made it possible to increase the number of meiotic COs (PCT application WO 2013/038376).

In continuing their research, the inventors have now identified another gene of which the inactivation produces effects similar to that of FANCM.

This is the “FIDGETIN” (FIDG) gene of Arabidopsis thaliana (AtFIDG): its inactivation compensates for the effects of that of AtMSH5, of SHOC1, of AtZIP4 or of AtHEI10 and makes it possible to increase the number of meiotic COs and to restore fertility in the zmm mutants Atzip4−/−, Atmsh5−/−, shoc1−/− and Athei10−/−. The inventors have also discovered that the effects of inactivation of the FIDG gene on the increase in the number of meiotic COs occurs not only in the zmm mutants, but also in wild-type plants which have functional ZMM genes, and that, when the inactivation of the FIDG gene is combined with that of the FANCM gene, the number of meiotic COs is further increased compared with those observed when these genes are inactivated separately. Moreover, no influence of FIDG gene inactivation on the somatic phenotype was observed.

The first fidg mutant was described as early as 1943 (Grüneberg, Journal of Genetics, 45, 22-28, 1943), as being associated in mice with a new phenotype comprising repeated shaking of the head and circular movements of the animal. Cox et al. (Nat Genet, 26, 198-202, 2000) identified the FIDGETIN gene, the mutation of which was responsible for this phenotype, and also discovered the existence in mice of 2 related genes which were called FIDGETIN-LIKE 1 and FIDGETIN-LIKE 2. Various functions of the FIDG proteins have been described, implicating them in particular in the regulation of osteoblast proliferation and differentiation (Park et al., J Bone Miner Res, 22, 889-96, 2007), the regulation of spermatogenesis (L'Hote et al., PLoS One, 6, e27582, 2011), microtubule severing (Mukherjee et al., Cell Cycle, 11, 2359-66, 2012), and participation in a complex, containing hsRAD51, involved in the repair of double-stranded breaks in somatic cells (Yuan & Chen, Proc Natl Acad Sci USA, 2013).

The FIDG proteins belonging to the family of AAA-ATPases (ATP-ases Associated with various Activities), and more particularly to subgroup 7 (Beyer, Protein Sci, 6, 2043-58, 1997; Frickey & Lupas, J Struct Biol, 146, 2-10, 2004). This subgroup also comprises the KATANIN and SPASTIN proteins which are, just like FIDGETIN, described as microtubule severing enzymes. They have been implicated in the regulation of microtubule number and size in many species: C. elegans (Yakushiji et al., FEBS Lett, 578, 191-7, 2004); D. melanogaster (Zhang et al., J Cell Biol, 177, 231-42, 2007); H. sapiens (Mukherjee et al., Cell Cycle, 11, 2359-66, 2012); A. thaliana, (Stoppin-Mellet et al., Biochem J, 365, 337-42, 2002).

The FIDG proteins appear to be conserved in animals and plants, but seem to be absent in yeasts. In animals, from one to three FIDGETIN-like proteins have been identified (in particular three in mammals); on the other hand, a single representative of this family has been identified in the genome of the nematode C. elegans, and of xenopus, and also in those of most plants.

Whether they are animal or vegetable, the FIDG proteins have two conserved protein domains: an AAA-ATPase domain, with a putative ATP hydrolysis role; and also a VSP4 domain, known to play an important role in binding to microtubules. These two domains are respectively listed under the references PF00004 and PF09336 in the PFAM database (Punta et al., Nucleic Acids Res. Database Issue 40:D290-D301, 2012).

The inhibition, in a plant, of a FIDG protein according to the present invention leads to an increase in meiotic CO frequency in said plant.

The sequence of the Arabidopsis thaliana FIDG protein determined by the inventors by sequencing the complementary DNA (cDNA) derived from the messenger RNA encoding FIDG, is represented in the appended sequence listing under number SEQ ID No. 1. The sequence of the corresponding FIDG gene is a composite sequence of the two overlapping genes listed in the TAIR10 database under the references AT3G27120 and AT3G27130. The messenger RNA is composed of 13 exons, the translation start being that of the gene predicted as AT3G27130 and the stop codon belonging to the gene predicted as AT3G27120 (the protein is encoded on the Crick strand). A poorly predicted splice site is responsible for the poor annotation of this gene in the databases.

A search in the sequence databases has made it possible to identify AtFIDG orthologs in a wide panel of eukaryotics, and it is very probable that this protein is conserved in all terrestrial plants. By way of nonlimiting example of AtFIDG orthologs, mention will in particular be made of:
the Brachypodium distachyon protein, the polypeptide sequence of which is available in the GenBank database under accession number XM_003576467;
the Carica papaya protein, the polypeptide sequence of which is predicted in the PLAZA database under accession number CP00171G00260;
the Fragaria vesca protein, the predicted polypeptide sequence of which appears in the Phytozome database under accession number mrna25885.1 and in the PLAZA database under accession number FV6G37220;
in Glycin max, two genes resulting from a duplication specific to this species exist; their polypetide sequence is available in the Phytozome database under accession numbers Glyma19g18350.2 and Glyma05g14440.2, and in the PLAZA database under the respective accession numbers GM19G18350 and GM05G14440;
the Oryza sativa protein, the polypeptide sequence of which is available in the GenBank database under the accession numbers ABA97741 or EEC69225.1, preferably EEC69225.1;
the Populus tricocarpa protein, the polypeptide sequence of which is available in the GenBank database under accession number POPTR_0001s33870;
the Ricinus communis protein, the polypeptide sequence of which is available in the GenBank database under accession number XM_002509479;
the Solanum lycopersicum protein, the polypeptide sequence of which is available in the GenBank database under accession number XM_004233540.1;
the Sorghum bicolor protein, the polypeptide sequence of which is available in the GenBank database under accession number XM_002442067.1;
the Theobroma cacao protein, the polypeptide sequence of which is predicted in the Phytozome database under accession number Thecc1EG017182t1 or Thecc1EG017182t2 (preferably Thecc1EG017182t2) and in the PLAZA database under accession number TC04G003320;
the Vitis vinifera protein, the polypeptide sequence of which is available in the GenBank database under accession number CBI21358;
the Zea mays protein, the polypeptide sequence of which is available in the GenBank database under accession number DAA54951;
the Hordeum vulgare protein, the polypeptide sequence of which is available in the GenBank database under accession number BAK02801.1;
the Triticum urartu protein, the polypeptide sequence of which is available in the GenBank database under accession number EMS65393.1.

A subject of the present invention is a method for increasing meiotic CO frequency in a plant, characterized in that it comprises the inhibition, in said plant, of a protein hereinafter called FIDG, said protein having at least 35%, and in increasing order of preference, at least 40, 45, 50, 55, 60, 65, 70, 75, 80, 85, 90, 95 or 98% sequence identity, or at least 50%, and in increasing order of preference, at least 50, 55, 60, 65, 70, 75, 80, 85, 90, 95 or 98% sequence similarity with the AtFIDG protein of sequence SEQ ID No. 1, and containing an AAA-ATPase domain (PF00004) and a VSP4 domain (PF09336).

Preferably, said protein has at least 70%, and in increasing order of preference at least 75, 80, 85, 90, 95 or 98% sequence identity, or at least 80%, and in increasing order of preference at least 85, 90, 95 or 98% sequence similarity, with any one of the AtFIDG orthologs of which the list is indicated above.

According to one preferred embodiment of the present invention, the AAA-ATPase domain of said FIDG protein has at least 60%, and in increasing order of preference at least 65, 70, 75, 80, 85, 90, 95 or 98% sequence identity, or at least 70%, and in increasing order of preference at least 75, 80, 85, 90, 95 or 98% sequence similarity, with the AAA-ATPase domain of the AtFIDG protein (amino acids 431-561 of SEQ ID No. 1).

According to another preferred embodiment of the present invention, the VSP4 domain of said FIDG protein has at least 60%, and in increasing order of preference at least 65, 70, 75, 80, 85, 90, 95 or 98% sequence identity, or at least 70%, and in increasing order of preference at least 75, 80, 85, 90, 95 or 98% sequence similarity, with the VSP4 domain of the AtFIDG protein (amino acids 623-672 of SEQ ID No. 1).

Advantageously, said FIDG protein also contains an RAD51-binding domain, having at least 50%, and in increasing order of preference at least 55, 60, 65, 70, 75, 80, 85, 90, 95 or 98% sequence identity, or at least 70%, and in increasing order of preference at least 75, 80, 85, 90, 95 or 98% sequence similarity, with amino acids 268-346 of SEQ ID No. 1.

Unless otherwise specified, the sequence identity and similarity values indicated herein are calculated over the whole of the length of the sequences compared, using the overall alignment algorithm of Needleman-Wunsch (Needle EMBOSS program with the default parameters). The similarity calculations are carried out using the BLOSUM62 matrix.

Inhibition can in particular be obtained by mutagenesis of the FIDG gene. For example, a mutation in the coding sequence can induce, depending on the nature of the mutation, the expression of an inactive protein, or of a protein with reduced activity; a mutation at a splice site can also impair or abolish the function of the protein; a mutation in the promoter sequence can induce the absence of expression of said protein, or a decrease in its expression.

The mutagenesis can be carried out, for example, by deletion of all or part of the coding sequence or of the promoter of FIDG, or by insertion of an exogenous sequence, for example a transposon or a T-DNA, within said coding sequence or said promoter. It can also be carried out by inducing point mutations, for example by EMS mutagenesis, by radiation, or by site-directed mutagenesis, for example using TALENs (Transcription Activator-Like Effector Nucleases) or zinc-finger nucleases (Christian et al., Genetics, 186, 757-61, 2010; Curtin et al., Plant Gen., 5, 42-50, 2012).

The mutated alleles can be detected for example by PCR, using primers specific for the FIDG gene.

Various high-throughput mutagenesis and screening methods are described in the prior art. By way of examples, mention may be made of methods of the “TILLING” (Targeting Induced Local Lesions In Genome) type, described by McCallum et al., Plant Physiol., 123, 439-442, 2000). The absence of FIDG functionality in the mutants can be verified on the basis of the phenotypic characteristics of their progeny; plants which are homozygotes for a mutation that inactivates the FIDG gene have a meiotic CO rate which is greater than that of the wild-type plants (not carrying the mutation in the FIDG gene) from which they are derived. Generally, this meiotic CO rate is greater by at least 50%, preferably at least 2 times greater than that of the wild-type plants from which they are derived.

Alternatively, the inhibition of the FIDG protein is obtained by silencing of the FIDG gene. Various techniques for gene silencing in plants are known in themselves (for review see, for example: Watson & Grierson, Transgenic Plants: Fundamentals and Applications (Hiatt, A, ed) New York: Marcel Dekker, 255-281, 1992; Chicas & Macino, EMBO reports, 21, 992-996, 2001). Mention may be made of antisense or cosuppression inhibition, described for example in patents U.S. Pat. No. 5,190,065 and U.S. Pat. No. 5,283,323. It is also possible to use ribozymes targeting the mRNA of the FIDG protein.

Preferably, the silencing of the FIDG gene is induced by RNA interference targeting said gene.

An interfering RNA (iRNA) is a small RNA which can silence a target gene in a sequence-specific manner.

Interfering RNAs comprise in particular “small interfering RNAs” (siRNAs) and micro-RNAs (miRNAs).

Initially, DNA constructs for expressing interfering RNAs in plants contained a fragment of 100 by or more (generally from 100 to 800 bp) of the cDNA of the targeted gene, under the transcriptional control of an appropriate promoter. Currently, the constructs most widely used are those which can produce a hairpin RNA transcript (hpRNA). In these constructs, the fragment of the target gene is inversely repeated, with generally a spacer region between the repeats (for review, cf. Watson et al., FEBS Letters, 579, 5982-5987, 2005). Use may also be made of artificial micro-RNAs (amiRNAs) directed against the FIDG gene (Ossowski et al., The Plant Journal, 53, 674-690, 2008; Schwab et al., Methods Mol Biol., 592, 71-88, 2010; Wei et al., Funct Integr Genomics., 9, 499-511, 2009).

A subject of the present invention is recombinant DNA constructs, and in particular expression cassettes, producing an iRNA which makes it possible to silence the FIDG gene.

An expression cassette in accordance with the invention comprises a recombinant DNA sequence of which the transcript is an iRNA, in particular an hpRNA or an amiRNA, targeting the FIDG gene, placed under the transcriptional control of a promoter which is functional in a plant cell.

A wide choice of promoters appropriate for the expression of heterologous genes in plant cells or plants is available in the art.

These promoters can be obtained for example from plants, plant viruses or bacteria such as Agrobacterium. They include constitutive promoters, namely promoters which are active in most tissues and cells and under most environmental conditions, and also tissue-specific or cell-specific promoters, which are active only or mainly in certain tissues or certain cell types, and inducible promoters which are activated by physical or chemical stimuli.

Examples of constitutive promoters which are commonly used in plant cells are the 35S promoter of the cauliflower mosaic virus (CaMV) described by Kay et al. (Science, 236, 4805, 1987), or derivatives thereof, the promoter of the cassava vein mosaic virus (CsVMV) described in International Application WO 97/48819, the maize ubiquitin promoter or the rice “actin-intron-actin” promoter (McElroy et al., Mol. Gen. Genet., 231, 150-160, 1991; GenBank accession number S 44221).

In the context of the present invention, use may also be made of a meiosis-specific promoter (i.e. a promoter which is active exclusively or preferentially in cells undergoing meiosis). By way of nonlimiting example, mention may be made of the DMC1 promoter (Klimyuk & Jones, Plant J., 11, 1-14, 1997).

The expression cassettes of the invention generally comprise a transcriptional terminator, for example the 3′NOS terminator of nopaline synthase (Depicker et al., J. Mol. Appl. Genet., 1, 561-573, 1982), or the 3′CaMV terminator (Franck et al., Cell, 21, 285-294, 1980). They can also comprise other transcription-regulating elements such as enhancers.

The recombinant DNA constructs in accordance with the invention also encompass recombinant vectors containing an expression cassette in accordance with the invention. These recombinant vectors can also include one or more marker genes, which allow the selection of the transformed cells or plants.

The choice of the most appropriate vector depends in particular on the intended host and on the method envisioned for the transformation of the host in question. Numerous methods for the genetic transformation of plant cells or of plants are available in the art, for many dicotyledonous or monocotyledonous plant species. By way of nonlimiting examples, mention may be made of virus-mediated transformation, microinjection-mediated transformation, electroporation-mediated transformation, microprojectile-mediated transformation, Agrobacterium-mediated transformation, etc.

A subject of the invention is also a host cell comprising a recombinant DNA construct in accordance with the invention. Said host cell may be a prokaryotic cell, for example an Agrobacterium cell, or a eukaryotic cell, for example a plant cell genetically transformed with a DNA construct of the invention. The construct can be expressed transiently; it can also be incorporated into a stable extrachromosomal replicon, or integrated into the chromosome.

The invention also provides a method for producing a transgenic plant having a meiotic CO rate which is higher than that of the wild-type plant from which it is derived, characterized in that it comprises the following steps:

transforming a plant cell with a vector containing an expression cassette in accordance with the invention;

culturing said transformed cell in order to regenerate a plant having in its genome a transgene containing said expression cassette.

The invention also encompasses plants genetically transformed with a DNA construct of the invention. Preferably, said plants are transgenic plants, in which said construct is contained in a transgene integrated into the genome of the plant, such that it is transmitted to the successive generations of plants. The expression of the DNA construct of the invention results in a downregulation of the expression of the FIDG gene, which confers on said transgenic plants a meiotic CO rate which is higher than that of the wild-type plants (not containing the DNA construct of the invention) from which they are derived. Generally, this meiotic CO rate is higher by at least 50%, preferably at least 2 times higher than that of the wild-type plants from which they are derived.

Particularly advantageously, the meiotic CO rate can be further increased by combining, in the same plant, the inhibition of the FIDG protein with that of the FANCM protein. In this case, the meiotic CO rate is at least 3 times higher, preferably at least 5 times higher than that of the wild-type plants from which they are derived.

The increase in the meiotic CO rate by inhibition of the FANCM protein is described in PCT application WO 2013/038376, the content of which is incorporated into the present description by way of reference.

The FANCM protein is defined as a protein having at least 30% and in increasing order of preference at least 35, 40, 45, 50, 55, 60, 65, 70, 75, 80, 85, 90, 95 or 98% sequence identity, or at least 45%, and in increasing order of preference at least 50, 55, 60, 65, 70, 75, 80, 85, 90, 95 or 98% sequence similarity, with the AtFANCM protein of Arabidopsis thaliana (GenBank: NP_001185141; UniProtKB: F4HYE4), and containing a DEXDc helicase domain (cd00046) and a HELICc helicase domain (cd00079). Preferably, the DEXDc helicase domain has at least 65%, and in increasing order of preference at least 70, 75, 80, 85, 90, 95 or 98% sequence identity, or at least 75%, and in increasing order of preference at least 80, 85, 90, 95 or 98% sequence similarity, with the DEXDc domain (amino acids 129-272) of the AtFANCM protein, and the HELICc helicase domain has at least 60%, and in increasing order of preference at least 65, 70, 75, 80, 85, 90, 95 or 98% sequence identity, or at least 70%, and in increasing order of preference at least 75, 80, 85, 90, 95 or 98% sequence similarity, with the HELICc domain (amino acids 445-570) of the AtFANCM protein. The polypeptide sequence of the AtFANCM protein is represented in the appended sequence listing under number SEQ ID No. 2.

The inhibition in a plant of a FANCM protein as defined above leads to an increase in meiotic CO frequency in said plant.

A subject of the present invention is therefore also:

a method for increasing the meiotic CO frequency in a plant, comprising the inhibition, in said plant, of the FIDG protein and of the FANCM protein;

expression cassettes and vectors comprising a recombinant DNA sequence of which the transcript is an interfering RNA targeting the FIDG gene, and a recombinant DNA sequence of which the transcript is an interfering RNA targeting the FANCM gene;

host cells and transgenic plants cotransformed with a recombinant DNA sequence of which the transcript is an interfering RNA targeting the FIDG gene, and a recombinant DNA sequence of which the transcript is an interfering RNA targeting the FANCM gene;

mutant plants containing, in the FIDG gene, a mutation which induces the inhibition of the FIDG protein, and, in the FANCM gene, a mutation which induces inhibition of the FANCM protein;

mutant and transgenic plants containing, in the FIDG gene, a mutation which induces inhibition of the FIDG protein, and transformed with a recombinant DNA sequence of which the transcript is an interfering RNA targeting the FANCM gene, or else containing, in the FANCM gene, a mutation which induces inhibition of the FANCM protein, and transformed with a recombinant DNA sequence of which the transcript is an interfering RNA targeting the FIDG gene.

The present invention can be applied in particular in the field of plant improvement, in order to accelerate the production of new varieties. It also makes it possible to facilitate recombination between related species, and therefore the introgression of characteristics of interest. It also makes it possible to accelerate the establishment of genetic maps and positional cloning.

The present invention applies to a wide range of monocotyledonous or dicotyledonous plants of agronomic interest. By way of nonlimiting examples, mention may be made of rapeseed, sunflower, potato, corn, wheat, barley, rye, sorghum, rice, soybean, bean, carrot, tomato, zucchini, pepper, eggplant, turnip, onion, pea, cucumber, leek, artichoke, beetroot, cabbage, cauliflower, lettuces, endive, melon, watermelon, strawberry plant, apple tree, pear tree, plum tree, poplar tree, vine, cotton plant, rose, tulip, etc.

The present invention will be understood more clearly by means of the additional description which follows, which refers to nonlimiting examples illustrating the effects of mutations of the AtFIDG gene on meiotic recombination and CO rate, and also to the appended figures:

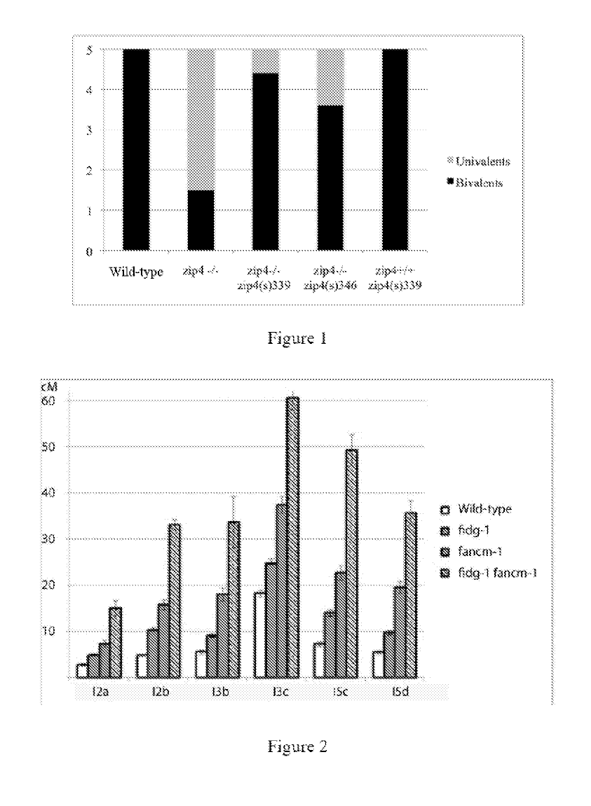

FIG. 1: Graph representing the number of bivalents in metaphase I of the suppressors (zmm—/— mutants) compared with that of wild-type plants and of homozygous zmm plants not mutated with EMS. Along the x-axis, phenotype of the plants tested; along the y-axis, number of bivalents (in black) and univalent pairs (in light grey) per meiosis.

FIG. 2: Graph representing the recombination frequencies measured in the fidg-1 fancm-1 double mutant.

### Legend:

□: Wild-type

[symbol] : fidg-1

[symbol] : fancm

[symbol] : fidg-1 fancm-1

Along the x-axis: intervals tested, according to the nomenclature of Berchowitz & Copenhaver (Nat. Protoc., 3, 41-50, 2008). I2a and I2b are two intervals adjacent on chromosome 2; I3b and I3c on chromosome 3; I5c and I5d on chromosome 5. Along the y-axis: genetic distance (in cM).

### EXAMPLE 1

### Production of ZMM Gene Mutation Suppressor Mutants

Arabidopsis thaliana seeds homozygous for T-DNA insertion into AtZIP4, SHOC1, AtHEI10, AtMSH5 or AtMSH4 (Atzip4−/−, shoc1−/−, Athei10−/−, Atmsh5−/− or Atmsh4−/−) were mutated using EMS (ethylmethane sulfonate). The plants derived from the mutated seeds (M1 population, heterozygous for the EMS-induced mutations, and homozygous for the mutation of the ZMM gene) have an identical phenotype, resulting from the inactivation of the ZMM gene, which is reflected by a large decrease in CO frequency, which leads to a large decrease in the number of bivalents, and a very sizable reduction in fertility (“semi-sterile” plants), resulting in the formation of short siliques which can be easily distinguished from those of the wild-type plants. The M1 plants were self-pollinated, so as to produce a population of descendants (M2 population) potentially containing plants homozygous for the EMS-induced mutations. Plants of the M2 population having siliques longer than those of the homozygous zmm plants of the M1 generation were selected and genotyped in order to verify their homozygous status with respect to the T-DNA insertion into the ZMM gene concerned. These are suppressors. The number of bivalents in metaphase I of these suppressors was compared with that of wild-type plants and of homozygous zmm plants not mutated with EMS.

The results are illustrated by FIG. 1.

While the wild-type plants systematically show 5 bivalents per meiosis, the zmm−/− mutants, in particular Atzip4−/− mutants, show a large decrease in the number of bivalents (and therefore the appearance of univalents). The mutations in FIDGETIN on an Atzip4−/− background (suppressors 339 and 346) make it possible to partially restore the formation of bivalents. The simple fidg mutants exhibit, like the wild-type, 5 bivalents.

In the zmm/SUPPRESSOR plants, poor chromosome segregation is observed, which results from a decrease in the number of bivalents, induced by the zmm mutation, and which results in nonviable unbalanced gametes. Conversely, in the zmm/suppressor plants, the number of bivalents is higher, ensuring a partly restored fertility. The ZMM/suppressor simple mutants behave like the wild-type plants; normal chromosome segregation is observed, resulting in the formation of balanced gametes.

The zmm/suppressor and ZMM/suppressor plants do not, moreover, exhibit any visible phenotypic somatic differences with the wild-type plants. The EMS-induced mutations are recessive. Indeed, they do not result in any detectable phenotype in the heterozygous state in the M1 mutant population, and the plants resulting from the backcrossing of the zmm/suppressor mutants with the initial zmm mutants from which they are respectively derived have a phenotype which does not differ from that of the initial mutants. Moreover, the segregation of the “fertile” and “semi-fertile” phenotypes in the descendants derived from the self-pollination of the plants resulting from the backcrossing of the zmm/suppressor mutants with the initial zmm mutants from which they are respectively derived indicates that the mutation concerns a single locus.

### EXAMPLE 2

### Localization in the Fidgetin Gene

Among the EMS mutants described in example 1 above, two lines of ZIP4 gene mutation suppressor mutants (FIG. 1), three lines of SHOC1 gene mutation suppressor mutants, three lines of AtMSH5 gene mutation suppressor mutants, three lines AtHEI10 gene mutation suppressor mutants, and also one line of AtMSH4 gene mutation suppressor mutants were isolated. These lines are respectively called: zip4 (s) 339, zip4 (s) 346, msh5 (s) 647, msh5 (s) 5, msh5 (s) 652, shoc1(s)101, shoc1(s)123, shoc1(s)161, hei10(s)141, hei10(s)208, hei10(s)235, msh4 (s) 80.

The corresponding mutations were all located in the genomic sequence of AtFIDGETIN. Complementation tests confirmed that these mutations were indeed responsible for the phenotype observed.

Table I below indicates the nature and the position of the mutations identified.

TABLE I

| | | Change in the | |
|---|---|---|---|
| | | nucleotide sequence | Change in the protein |
| FIDG alleles | | (TAIR 10) | sequence |
| zip4(s)339 | fidg-2 | Chr3 10 001 129: C > T | Modified splice site |
| zip4(s)346 | fidg-1 | Chr3 10 001 783: | Frame shift, STOP |
| | | deletion 1 bp | |
| msh5(s)647 | fidg-3 | Chr3 10 000 278: C > T | STOP |
| msh5(s)5 | fidg-4 | Chr3 10 004 090: C > T | STOP |
| msh5(s)652 | fidg-5 | Chr3 10 001 772: C > T | Amino acid change: |
| | | | E > K |
| shoc1(s)101 | fidg-6 | Chr3 10 001 017: G > A | Amino acid change: |
| | | | L > F |
| shoc1(s)123 | fidg-7 | Chr3 10 001 812: C > T | Modified splice site |
| shoc1(s)161 | fidg-8 | Chr3 10 001 909: G > A | Amino acid change: |
| | | | R > K |
| hei10(s)141 | fidg-9 | Chr3 10 001 970: C > T | STOP |
| hei10(s)208 | fidg-10 | Chr3 10 000 328: C > T | Amino acid change: |
| | | | E > K |
| hei10(s)235 | fidg-11 | Chr3 10 001 422: C > T | Amino acid change: |
| | | | D > N |
| msh4(s)80 | fidg-12 | Chr3 10 000 840: C > T | Amino acid change: |
| | | | L > F |

### EXAMPLE 3

### Influence of the Inactivation of the Fidg Gene on Meiotic Recombination Frequency

The meiotic recombination frequencies in fidg mutant plants and wild-type plants were compared. The meiotic recombination frequency was measured by tetrad analysis using fluorescent labels, as described by Berchowitz & Copenhaver (Nat. Protoc., 3, 41-50, 2008). The genetic distance was measured on 2 adjacent intervals on chromosome 2 (I2a and I2b), 2 adjacent intervals on chromosome 3 (I3b and I3c), and also 2 adjacent intervals on chromosome 5 (intervals I5c and I5d). The results are illustrated by FIG. 2 and table II.

The mutation of the FIDGETIN gene increases recombination compared with the wild-type by on average 72% over the 6 intervals.

### EXAMPLE 4

### Influence of the Combined Inactivation of the Fidg and Fancm Genes on Meiotic Recombination Frequency

In the same way as in example 2, the recombination frequencies were measured in the fidg-1 fancm-1 double mutant. The results are represented in FIG. 2 and in table II.

Table II represents the genetic sizes (in cM) of the various intervals in the various mutants (the standard errors are indicated between parentheses; ND: not determined).

TABLE II

| | wild- | | | | | | fidg-1 | |
|---|---|---|---|---|---|---|---|---|
| cM | type | +/− | fidg-1 | +/− | fancm-1 | +/− | fancm-1 | +/− |
| I2a | 2.86 | 0.26 | 4.91 | 0.29 | 7.41 | 0.73 | 15.10 | 1.68 |
| I2b | 4.82 | 0.20 | 10.33 | 0.52 | 15.82 | 0.99 | 33.17 | 1.09 |
| I3b | 5.62 | 0.36 | 9.20 | 0.37 | 18.07 | 1.40 | 33.75 | 5.46 |
| I3c | 18.30 | 0.73 | 24.81 | 0.96 | 37.44 | 1.83 | 60.60 | 2.69 |
| I5c | 7.35 | 0.53 | 14.08 | 0.75 | 22.72 | 1.55 | 49.32 | 3.36 |
| I5d | 5.54 | 0.37 | 9.80 | 0.53 | 19.63 | 1.33 | 35.70 | 2.61 |
| I2a | 2.8 | (0.2) | 4.9 | (0.3) | 9.0 | (0.7) | ND | |
| I2b | 4.8 | (0.3) | 9.4 | (0.5) | 17.1 | (1.0) | ND | |
| I3c | 5.8 | (0.5) | 9.7 | (0.7) | 15.4 | (1.1) | ND | |
| I3d | 19.7 | (1.1) | 24.2 | (1.2) | 34.5 | (2.0) | ND | |
| I5c | 6.2 | (0.1) | 9.6 | (0.5) | 17.8 | (0.6) | 35.7 | (2.6) |
| I5d | 6.3 | (0.1) | 13.5 | (0.8) | 21.5 | (0.8) | 49.3 | (3.4) |

The results show that the joint inactivation of FIDG and FANCM (fidg-1 fancm-1) results in an increase in recombination of on average 5.77 times compared with the wild-type (mean over the 6 intervals).

1. A method for increasing meiotic CO frequency in a plant, characterized in that it comprises the inhibition, in said plant, of a protein hereinafter called FIDG, said protein having at least 45% sequence identity, or at least 60% sequence similarity, with the AtFIDG protein of sequence SEQ ID No. 1, and containing an AAA-ATPase domain and a VSP4 domain.

2. The method as claimed in claim 1, characterized in that it also comprises the inhibition, in said plant, of a protein hereinafter called FANCM, said protein having at least 30% sequence identity, or at least 45% sequence similarity, with the AtFANCM protein of sequence SEQ ID No. 2, and containing a DEXDc helicase domain and a HELICc helicase domain.

3. The method as claimed in claim 1, characterized in that the inhibition of the FIDG protein is obtained by mutagenesis of the gene encoding said protein or of its promoter.

4. The method as claimed in claim 1, characterized in that the inhibition of the FIDG protein is obtained by silencing of the gene encoding said protein by expressing, in said plant, an interfering RNA targeting said gene.

5. An expression cassette, characterized in that it comprises a recombinant DNA sequence of which the transcript is an interfering RNA targeting the FIDG gene, placed under the transcriptional control of a promoter which is functional in a plant cell.

6. The expression cassette as claimed in claim 5, characterized in that it also comprises a recombinant DNA sequence of which the transcript is an interfering RNA targeting the FANCM gene, placed under the transcriptional control of a promoter which is functional in a plant cell.

7. A recombinant vector containing an expression cassette as claimed in claim 5.

8. A host cell containing a recombinant DNA sequence of which the transcript is an interfering RNA targeting the FIDG gene, placed under the transcriptional control of a promoter which is functional in said cell.

9. The host cell as claimed in claim 8, characterized in that it also contains a recombinant DNA sequence of which the transcript is an interfering RNA targeting the FANCM gene, placed under the transcriptional control of a promoter which is functional in said cell.

10. A mutant plant characterized in that it contains a mutation in the FIDG gene, said mutation inducing inhibition of the FIDG protein in said plant.

11. The mutant plant as claimed in claim 10, characterized in that it also contains a mutation in the FANCM gene, said mutation inducing inhibition of the FANCM protein in said plant.

12. The mutant plant as claimed in claim 10, characterized in that it also contains a recombinant DNA sequence of which the transcript is an interfering RNA targeting the FANCM gene, placed under the transcriptional control of a promoter which is functional in said plant.

13. A transgenic plant containing a transgene comprising a recombinant DNA sequence of which the transcript is an interfering RNA targeting the FIDG gene, placed under the transcriptional control of a promoter which is functional in said plant.

14. The transgenic plant as claimed in claim 13, characterized in that it also contains a recombinant DNA sequence of which the transcript is an interfering RNA targeting the FANCM gene, placed under the transcriptional control of a promoter which is functional in said plant.

15. The transgenic plant as claimed in claim 13, characterized in that it also contains a mutation in the FANCM gene, said mutation inducing inhibition of the FANCM protein in said plant.

16. The method as claimed in claim 2, characterized in that the inhibition of the FIDG protein and of the FANCM protein is obtained by mutagenesis of the gene encoding said protein or of its promoter.

17. The method as claimed in claim 2, characterized in that the inhibition of the FIDG protein and of the FANCM protein is obtained by silencing of the gene encoding said protein by expressing, in said plant, an interfering RNA targeting said gene.

18. A recombinant vector containing an expression cassette as claimed in claim 6
